(19) United States
(12) Patent Application Publication
(10) Pub. No.: US 2023/0067926 A1
(43) Pub. Date: Mar. 2, 2023

(54) SYSTEMS AND METHODS FOR MONITORING PLATELET ACTIVATION

(71) Applicant: The Regents of the University of Michigan, Ann Arbor, MI (US)

(72) Inventors: Terry Major, Ann Arbor, MI (US); Deokwon Seo, Ann Arbor, MI (US); Jinsang Kim, Ann Arbor, MI (US)

(21) Appl. No.: 17/868,422

(22) Filed: Jul. 19, 2022

Related U.S. Application Data
(60) Provisional application No. 63/223,674, filed on Jul. 20, 2021.

(51) Int. Cl. G01N 33/86; C07K 16/18

(52) U.S. Cl. CPC G01N 33/86; C07K 16/18; G01N 2800/224; G01N 2333/75

(57) ABSTRACT

Provided herein are compositions, systems, and methods for monitoring platelet activation. In particular, provided herein are sensor devices for measuring platelet activation and uses thereof.

This application claims priority to U.S. Provisional Patent Application No. 63/223,674, filed Jul. 20, 2021, the entire contents of which are incorporated herein by reference for all purposes.

### GOVERNMENT SUPPORT

This invention was made with government support under HL111213 and EB016236 awarded by the National Institutes of Health. The government has certain rights in the invention.

### FIELD OF THE DISCLOSURE

Provided herein are compositions, systems, and methods for monitoring platelet activation. In particular, provided herein are sensor devices for measuring platelet activation and uses thereof.

### BACKGROUND OF THE DISCLOSURE

Heart disease and its related symptoms affect hundreds of thousands of Americans each year. According to the Center for Disease Control and Prevention, heart disease remains the leading cause of death in the United States.

For many patients, taking antiplatelet medication, Aspirin, Plavix, and Tirofiban, has been a commonly recommended practice to help prevent heart attacks and strokes. This family of antiplatelet drugs is commonly used to prevent blood clotting in patients with cardiovascular conditions such as myocardial infarction, stroke, and prosthetic heart valve issues. However, medicating antiplatelet drugs is often hazardous since it can lead to haphazard bleeding upon accident or surgery, which may require an emergency operation. In addition, many medical symptoms such as sepsis, malignancies, extracorporeal circulation, and dialysis may cause abnormal platelet activity. Continuous monitoring of platelet function from patients with cardiac records is critical as many patients with the record may adjust dosage arbitrarily or take multiple medications from other diseases. However, monitoring of platelet function has been crudely implemented by either measuring bleeding time from a wound or by analyzing blood samples using complicated methods such as aggregometry,¹ cytometry,² or atomic force microscope (AFM).³ Although the latter provides accurate results, it complicates the monitoring too much for the patients since it requires access to a laboratory, too long examination time and costs. Consequently, patients are often simply advised to take the standard dose without accurate and vital monitoring of platelets; a practice that may endanger the lives of the patients.

What is needed are improved methods for monitoring platelet activation.

### SUMMARY OF THE DISCLOSURE

Provided herein are compositions, systems, and methods for monitoring platelet activation. In particular, provided herein are sensor devices for measuring platelet activation and uses thereof. The devices and methods described herein provide an easy to use, low cost, quantitative or semi-quantitative assay for platelet activation has been needed for a long time and did not exist prior to the present disclosure.

Experiments described herein developed a PDA-based microarray sensor device as an effective tool to specifically determine the level of activated platelet in whole blood samples using antibodies (e.g., 9F9 antibodies) that are specific for fibrinogen bound to the activated platelets tethered at the PDA liposome surface to provide selective recognition of activated platelet. The fluorescence signal intensity inversely related to the amount of Tirofiban, an inhibitor of platelet activated coagulation, added to whole blood samples, allowing for determination of appropriate dosage of antiplatelet drugs. Without any special separation process or pretreatment of whole sample, patients as well as their healthcare providers can conveniently monitor the level of activated platelet as a reference for appropriate anti-platelet drug dosage.

For example, in some embodiments, provided herein is a method, comprising: a) contacting a sample comprising platelets with a device comprising a solid surface comprising an array of polydiacetylene (PDA), and optionally liposomes conjugated to an antibody that specifically binds to activated platelets; and b) detecting activated platelets bound to the antibody.

Further embodiments provide the use of a device comprising a solid surface comprising an array of polydiacetylene (PDA)-liposomes conjugated to an antibody that specifically binds to activated platelets to detect activation platelets in a blood sample.

Also provided is a device comprising a solid surface comprising an array of polydiacetylene (PDA)-liposomes conjugated to an antibody that specifically binds to activated platelets.

The present disclosure is not limited to particular PDA or liposomes. In some exemplary embodiments, the PDA is 10,12-pentacosadiynoic acid (PCDA)-epoxy and the liposome comprises 1,2-dimyristoyl-sn-glycero-3-phosphate (DMPA).

In some embodiments, the solid support is glass (e.g., amine modified or other modification).

Any antibody that is specific for activated platelets is suitable for use in the devices and methods described herein. In some embodiments, the antibody binds to fibrinogen on activated platelets. Examples of suitable antibodies include but are not limited to, 9F9. In some embodiments, the antibody is unlabeled. In some embodiments, the antibody is labeled with an exogenous label (e.g., with a detectable label such as FITC).

In some embodiments, the detecting comprises detecting fluoresce emitting from said device (e.g., due to structural changes in the PDAs). In some embodiments, the detecting is quantitative. For example, in some embodiments, the quantitative detecting comprises comparing the level of fluorescence to a standard level (e.g., indicative of a threshold level of activation of said platelets).

In some embodiments, the blood sample is from a subject. In some embodiments, the blood sample is whole blood or a blood product. In some embodiments, the subject has been administered an anti-platelet drug. In some embodiments, the detecting is repeated one or more times during the administration. In some embodiments, the detecting is used to determine a treatment course of action (e.g., including but not limited to, changing the dose of the anti-platelet drug, changing the anti-platelet drug, and stopping the anti-platelet drug). In some embodiments, the anti-platelet drug is a test compound.

Certain embodiments provide a method of contacting the blood sample with an anti-platelet drug prior to said detecting (e.g., in vitro), for example, to screen candidate anti-platelet drugs or determine a dosage of an anti-platelet drug.

Additional embodiments are described herein.

### DESCRIPTION OF THE DRAWINGS

FIG. 1. (A) Absorption and emission spectra of the blue and red phases of PCDA-epoxy liposome tethered on an amine-functionalized glass substrate. Fluorescence microscopy images of (B) a negative control PDA device without antibody incubation, a PDA microarray device having 9F9 antibodies without (C) and with (D) ethanolamine blocking, (E) fluorescence emission intensity of the PDA liposome of each sample arrays.

FIG. 2. (A) Fluorescence microscope images and (B) fluorescence emission intensity of PDA liposome arrays after 20-min incubation at room temperature. Scale bars are 500 μm.

FIG. 3. Fluorescence intensity reduction ratio of PDA liposome-9F9 microarray devices after 20-min incubation with Tirofiban and whole blood. (inset) Fluorescence microscopy images.

FIG. 4(A-F) FACS analysis of the FITC-9F9 antibody in human whole blood, (G) Flow cytometric analysis of collagen concentration response on 9F9 antibody expression. MFI=maximum fluorescence intensity, (H) concentration response of GPIIb/IIIa inhibitor, Tirofiban, on collagen and epinephrine stimulated human platelets.

FIG. 5. Schematic illustration of (A) PDA liposome self-assembly, photopolymerization, and stimuli-responsive property, (B) platelet activation assay based on sensory PDA liposome-9F9 antibody.

FIG. 6 Chemical structure of PDA-epoxy and DMPA, (B) Schematic illustration of the PDA liposome microarray fabrication procedure for detecting platelet activation.

### Definitions

To facilitate an understanding of the present disclosure, a number of terms and phrases are defined below:

The term “sample” is used in its broadest sense. On the one hand it is meant to include a specimen or culture. On the other hand, it is meant to include both biological and environmental samples. A sample may include a specimen of synthetic origin.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

### DETAILED DESCRIPTION OF THE DISCLOSURE

Provided herein are compositions, systems, and methods for monitoring platelet activation. In particular, provided herein are sensor devices for measuring platelet activation and uses thereof.

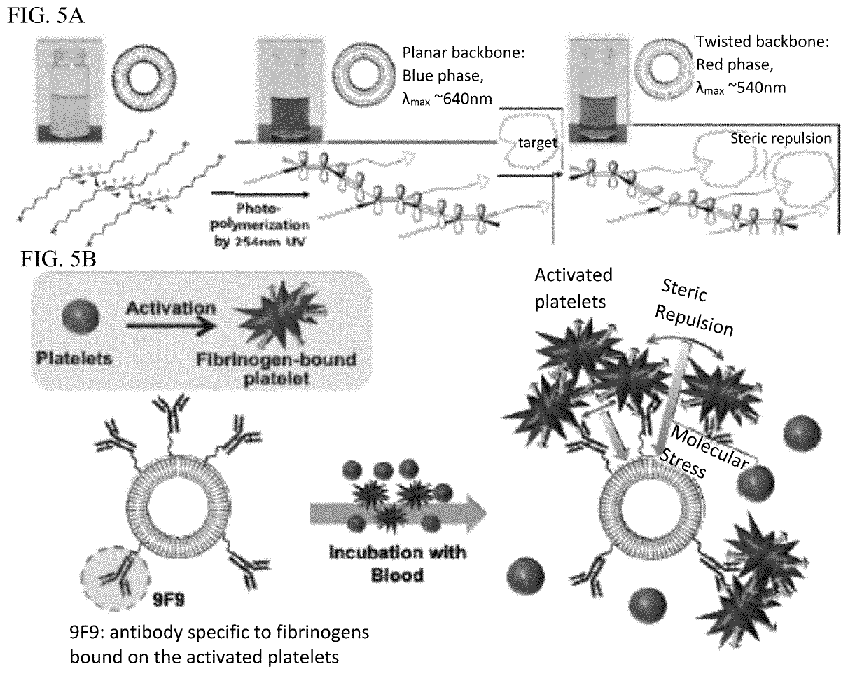

Polydiacetylene (PDA) is a unique sensory material which changes its optical properties in response to molecular stress produced by steric repulsion between the captured analytes on adjacent receptors, providing useful self-signaling optical detection as illustrated in FIG. 5.⁴⁻⁶ Upon exposure to various molecular stresses, the color of PDA is changed from blue to red through the distortion of its conjugated yne-ene main chain and a consequential bandgap change. Furthermore, the converted red-phase PDA also emits red fluorescence while the original blue-phase PDA has no fluorescence emission. Through such a dual colorimetric and fluorometric self-signaling property, the PDA has been used to conveniently and sensitively detect certain molecular stresses generated by heat,⁷⁻⁹ humidity,^{10,11} and binding of metal ions,¹²⁻¹⁴ certain chemicals,¹⁵⁻²¹ or certain biomolecules²²⁻²⁸. A biosensor platform using PDA materials has been described.^{10, 12-14, 17, 22, 24, 29} In addition, an amphiphilic PDA monomer having an epoxy headgroup and self-assembled them into the liposome form has been developed.¹⁴ The resulting PDA-epoxy liposomes were efficiently tethered to amine-functionalized substrates by amine-epoxy chemistry, rendering practical microarray-type of PDA liposome biosensors.

In some embodiments, this sensory PDA platform is modified and used as an activated platelet detection system by employing an activated platelet specific antibody (e.g., 9F9 antibody), to provide selectivity (FIG. 5B).^{30, 31} In experiments described herein, the developed PDA liposome-9F9 microarray produced an intense fluorescence signal when the platelets in whole blood were activated and also could distinguish reduced platelet activation when varying amounts of an antiplatelet drug, Tirofiban, were added to blood samples by the signal intensity change. The presented results of this single-step bench-top assay provide a practical test that processes a large number of samples economically and to derive test results in a timeframe meaningful for clinical outcomes in acute situations such as angina pectoris, stroke, peripheral vascular disease or procedures involving angioplasty or coronary thrombolysis. The assay incorporates simple, sensitive and rapid attributes that can detect the extent of platelet activation.

The PDA liposome assays of embodiments of the present disclosure allow 1) a rapid and simple measurement using whole blood samples without the need for expensive and complicated blood cell separation equipment as well as the need for additional in vitro platelet stimulation with exogenous agonists such as ADP or collagen and 2) a sensitive assessment of various degrees of platelet activation especially the determination of platelet function of patients on low dose aspirin or of patients on antiplatelet drugs.

The present disclosure is not limited to particular PDA or liposomes. In some exemplary embodiments, the PDA is 10,12-pentacosadiynoic acid (PCDA)-epoxy and the liposome comprises 1,2-dimyristoyl-sn-glycero-3-phosphate (DMPA).

In some embodiments, the solid support is glass (e.g., amine modified).

Any antibody that is specific for activated platelets is suitable for use in the devices and methods described herein. In some embodiments, the antibody binds to fibrinogen on activated platelets. In some embodiments, the antibody is an antibody that recognizes human fibrinogen bound to its receptor CD41/CD61 (GPIIbIIIa, 61Ib73), bound to plastic surface or cleaved by plasmin (D fragment). In some embodiments, the antibody binds to epitopes located in the NH₂-terminal part of the γ-chain of fragment D (γ 112-119). Examples of suitable antibodies include but are not limited to, 9F9. In some embodiments, antibodies are labeled (e.g., with FITC) or unlabeled.

Exemplary PDA-liposome microarrays are described, for example, in U.S. Pat. Nos. 8,633,140 and 9,523,683; each of which is herein incorporated by reference in its entirety. Polydiacetylenes (PDA) are conjugated polymers that may include a backbone of alternating double and triple bonds and may be formed from the 1,4-addition polymerization of 1,3-diacetylenes, for example. Polydiacetylenes generally absorb well in the visible region of the electromagnetic spectrum, and hence are highly colored, ranging from blue to yellow. Diacetylene monomers can form various ordered systems, including crystals, liquid crystals, liposomes, films, and wires that are polymerized to form a polymer. Liposomes can be made from monomers with diacetylene chains and polar head groups; e.g., phosphatidylcholines and analogues thereof. The liposomes can be polymerized with UV light or gamma-radiation. Monomer films can be formed by Langmuir-Blodgett methods or cast from solvents and polymerized with UV light or gamma-radiation. The choice of monomer structure, conditions of liposome or film formation, and polymerization conditions can each affect the conjugation length of the polydiacetylene backbone, and hence the color of the system.

Upon heating, for example, the polymerized PDA system can undergo a change in the effective conjugation length, from the longer length forms (blue and purple) to the shorter length forms (red and yellow). This change may be attributed to the side-chains moving and repacking upon being heated. Soluble polydiacetylenes show solvo-chromic behavior and polydiacetylene films often change color upon exposure to solvent vapors. Polydiacetylene films and liposomes formed from diacetylene surfactants also often change color with a change in pH. In the case of packed polymer arrays that form films and liposomes, changes in the environment that affect the organization and packing of the side chains off the conjugated backbone can affect the conjugation length and hence the chromic and electronic properties of the polymer.

In some embodiments, the sensor comprises a hydroxyphenyl-benzoxazole (HBO) derivative and a plurality of diacetylene monomer. At least a portion of the diacetylene monomer is coupled to one or more affinity components having affinity for one or more respective targets (e.g., activated platelets). The sensor exhibits a change in fluorescence or conductivity when an affinity component interacts with a respective target. For example, an affinity component may be an antibody and a target may be an antigen.

The present sensors can be incorporated into microarrays. For example, microarrays are solid substrates composed of immobilized antibodies, aptamers, or peptides serving as probing molecules for the detection of specific targets. The present sensors may be used to generate a sensitive and label-free sensory signal at a binding recognition event that is readily applicable to the detection of many different proteins.

The PDA-based sensors described herein find use in the detection of platelet activation in research, screening, and clinical applications.

In some embodiments, the detecting is quantitative. For example, in some embodiments, the quantitative detecting comprises comparing the level of fluorescence to a standard level (e.g., indicative of a threshold level of activation of said platelets). In some embodiments, the level is compared to a standard level indicative of a safe or acceptable level of platelet activation.

For example, in some embodiments, assays are performed to quantitate platelet activation in a subject undergoing therapy with an anti-platelet drug (e.g., including but not limited to Aspirin, Plavix, or Tirofiban). In some embodiments, the detecting is used to determine a treatment course of action (e.g., including but not limited to, changing the dose of the anti-platelet drug, changing the anti-platelet drug, and stopping the anti-platelet drug).

In some embodiments, the devices described herein find use in screening (e.g., drug screening) and research uses. For example, platelet activation can be measured in a subject enrolled in a clinical study to determine if a drug alters platelet activation or to screen anti-platelet drugs. In some embodiments, assays are used to determine an appropriate dose of an anti-platelet drug.

In some embodiments, an in vitro assay is performed in which a blood sample is contacted with a test compound in vitro prior to determining platelet activation levels.

Experimental

### Example 1

Methods

(PCDA)-epoxy was synthesized as described.¹⁴ A phospholipid, 1,2-dimyristoyl-sn-glycero-3-phosphate (DMPA) was ordered from Avanti Polar Lipids. FITC-9F9 Antibody, Collagen, and Tirofiban were purchased from Biocytex, Chrono-log, and Sigma-Aldrich, respectively. Solvents, buffers, and blocking agents were purchased from Sigma-Aldrich.

PDA-Epoxy Liposome Assembly. The PDA liposomes comprising PCDA-epoxy and DMPA lipids were assembled by the following injection method.¹⁴ PCDA-epoxy and DMPA were co-dissolved (4:1 molar ratio) in 0.1 ml of tetrahydrofuran/water mixture (9:1 v/v) and the lipid solution was injected to the 20 ml of 5 mM HEPES buffer at pH 8. The total lipid concentration in the final aqueous solution was 0.5 mM. The liposome solution was probe-sonicated with 120 W for 10 mins and was filtrated through a 0.8 μM cellulose acetate syringe filter. The filtrate was stored at 5° C. before use.

PDA Liposome Microarray Fabrication. The surface of the glass was treated with 3-aminopropyltriethoxysilan to make amine functionality following a described process.′ A slight modification was made when immobilizing PDA liposome on the resulting amine-modified glass substrate and the details are as follows. Glass slides were washed with chloroform, acetone, and 2-propanol for 15 mins each. The cleaned glass slides were then sonicated in sulfuric acid containing NOCHROMIX for 2 hours. After a thorough rinse with deionized water and air drying, the glass slides were placed in a UV/Ozone cleaning apparatus and treated for 30 mins. The glass slides were then stirred in a 2 wt % 3-aminopropyltriethoxysilane in anhydrous toluene solution on an orbital shaker for 6 hrs in a glove box at 70° C. and afterward baked at 115° C. for 30 mins. The glass slides were sonicated in toluene, toluene:methanol (1:1), and methanol for 15 mins each to remove any unbound silane reagent.

To fabricate the PDA liposome Microarray, the liposomes were covalently immobilized on amine-modified glass slides through the amine-epoxy chemistry. 0.5 mM PDA-epoxy liposome solution was spotted on amine-coated glass slides with a manual microarrayer (VP 475, V&P scientific, INC) and was incubated at 5° C. for 24 hrs to prevent fast drying of the spotted solution. After removing the unbound liposomes by rinsing with 5 mM of pH 8 HEPES buffer, the PDA liposome-spotted slides were incubated with 0.06 mg/ml of 9F9 antibody in 5 mM of pH 8 HEPES buffer for 1 hr. After removing the unbound antibodies by rinsing with 5 mM of pH 8 HEPES buffer. The polymerization of the immobilized PDA-epoxy liposomes on the slides was carried out by illuminating 254 nm UV (1 mW/cm²) for 1 minute. The resulting PDA liposome microarray was used for subsequent detection analysis.

Detection of Activated Platelet with PDA Liposome Microarray. Samples were taken from healthy blood donors who had abstained from taking aspirin for two weeks before sampling. Blood samples were taken by median venipuncture into sodium citrate tube (BD Vacutainer) and were used immediately. Platelet-free plasma was obtained from the supernatant after 15-min centrifugation of the blood at 1500 G and was used immediately. For the subsequent experiments, 4 ml of collagen solution (1 mg/ml) and 2 ml of various concentration of Tirofiban solution were added to the 94 ml of blood samples, and the mixture was loaded immediately on the PDA liposome microarray. After 20 mins of incubation, fluorescence microscope images were obtained on Olympus BX 71 microscope equipped with a mercury lamp and a cut-off filter of 540 nm excitation and 600 nm emission. For fluorescence signal intensity measurement, the fabricated PDA liposome-9F9 microarray was placed on top of a 96-well plate and the combined substrates were inserted into a plate reader. Fluorescence emission intensities at 634 nm of total 120 microarray spots from three different devices were individually measured by using the excitation wavelength of 548 nm.

Fluorescence-activated Cell Sorting Analysis. Platelets diluted from whole blood (1:100) were treated with Tirofiban (0-10 nM), GPIIb/IIIa fibrinogen receptor inhibitor, and incubated for 20 min at room temperature. In a separate tube, 1 μl 10% DMSO was added to serve as a vehicle control (i.e., 0 nM Tirofiban). Collagen (40 μg/ml) was then added (4 μl) and incubated for 2 min at room temperature in all tubes. Platelet activation was measured by using 20 μl of the anti-9F9 antibody-FITC and 10 μl of the constitutive platelet marker, anti-CD61-PE (Phycierythrin). As a control for the anti-9F9 antibody, 10 μl of the isotype control, anti-mouse IgG1-FITC, was used with 10 μl of anti-CD61-PE in a separate tube. The antibodies were incubated at room temperature in the dark for 15 minutes. After the incubation, 700 μl of 1% formalin/dPBS was added to each tube and stored at 4° C. for up to 24 hrs post fixing until ready to run the fluorescence-activated cell sorting (FACS) analysis on a FACSCalibur flow cytometer (Beckton Dickenson San Jose, Calif.). CellQuest software (Beckton Dickenson San Jose, Calif.) was employed for the data analysis.

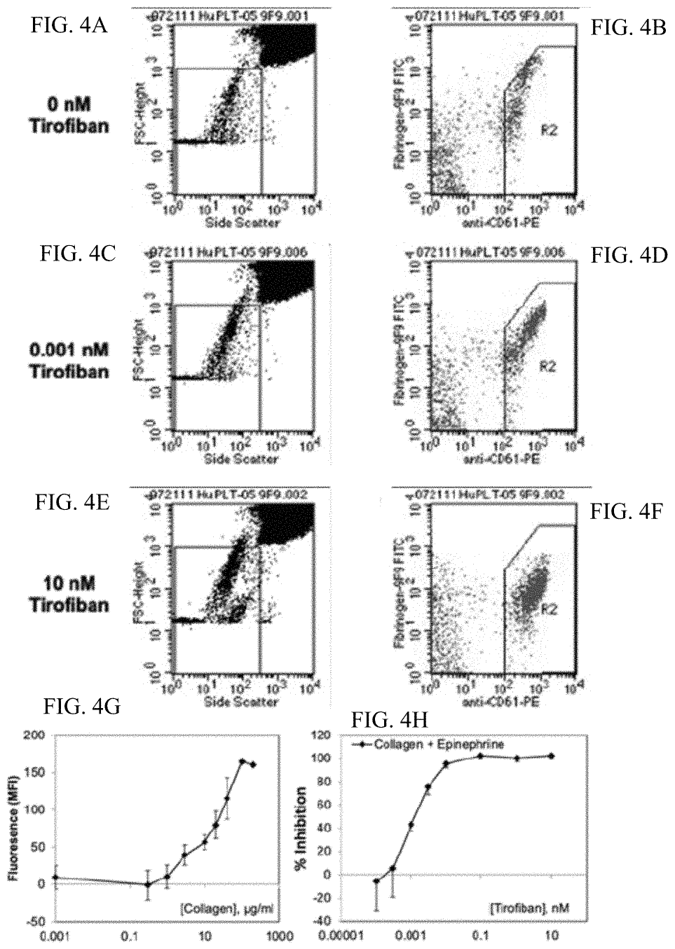

Cell populations were identified for data collection by their log forward scatter (FSC) and log side scatter (SSC) light profiles. For each sample, 5,000 total events were collected in a region (R1) gated on platelets within the FSC versus SSC profile (FIGS. 4A, C, E). Mean fluorescence intensity (MFI) of the anti-9F9 immunostaining was quantitated by a FITC (FL1) versus PE (FL2) log plot analysis. The platelets from region R1 were plotted and a gated region R2 was drawn to separate the platelets from debris on the FL2 axis (FIGS. 4B, D, and F). At this point all tubes were run on the cytometer noting the WI change in the platelet cloud in region R2. The adjusted WI was expressed as the geometric mean channel fluorescence minus the appropriate isotype control. The platelet reactivity index (PRI) was calculated by the following equation:

PRI=[(MFI_{c(T1)}−MFI_{C(T2)})/MFI_{C(T1)}]×100 (1)

where, MFI_{c(T1)} is the adjusted MFI for the collagen plus vehicle control tube, and the MFI_{c(T2)} is the adjusted MFI for the collagen plus each concentration of Tirofiban tubes.

Results

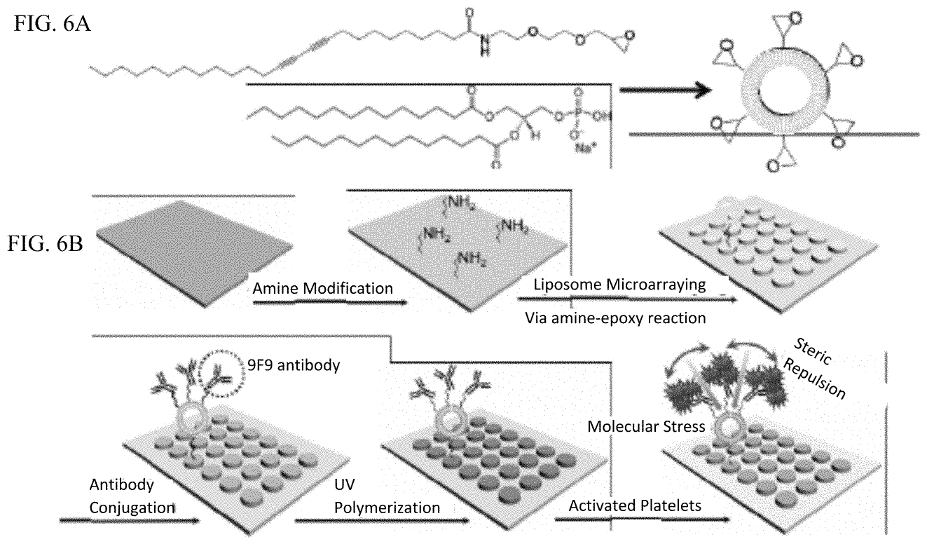

The overall fabrication procedure of the developed PDA liposome-9F9 antibody microarray is schematically illustrated in FIG. 6. Solid-state biosensors were used to directly use a whole blood sample without pre-treatment or pre-separation. The predominant reason for pretreating blood samples lies in the fact that whole blood is comprised of non-specific components such as red blood cell, white blood cell, and inactivated platelets that may interfere with the optical detection through colorimetric change. To circumvent this problem a separation process is usually required—centrifugation or filtration, especially as seem in the solution-type detection system. However, this makes the sensor cumbersome to use and significantly lengthens the time for detection or data acquisition. A PDA liposome was constructed by using (10,12-pentacosadiynoic acid)-epoxy (PCDA-epoxy) and 1,2-dimyristoyl-sn-glycero-3-phosphate (DMPA) as shown in FIG. 6. PDCA-epoxy monomer was designed for the following two reasons. First, the epoxy group allows the epoxy-amine chemistry between the PDA liposome and the amine-modified glass substrate that enables stable and covalent immobilization of PDA liposome onto the glass substrate.^{14, 24} This was further beneficial for binding of 9F9 antibodies to the PDA liposome surface as one can use the same epoxy-amine chemistry between the epoxy group on the PDA-epoxy and the amine groups of the antibody. A phospholipid DMPA, another constituent for self-assembling PDA liposome, was used to alter the self-assembly of the PDA monomer and to prevent high-order packing. Such insertion of phospholipid increased the flexibility of the liposome bilayer, which led to increased device sensitivity.^{17,22,24}

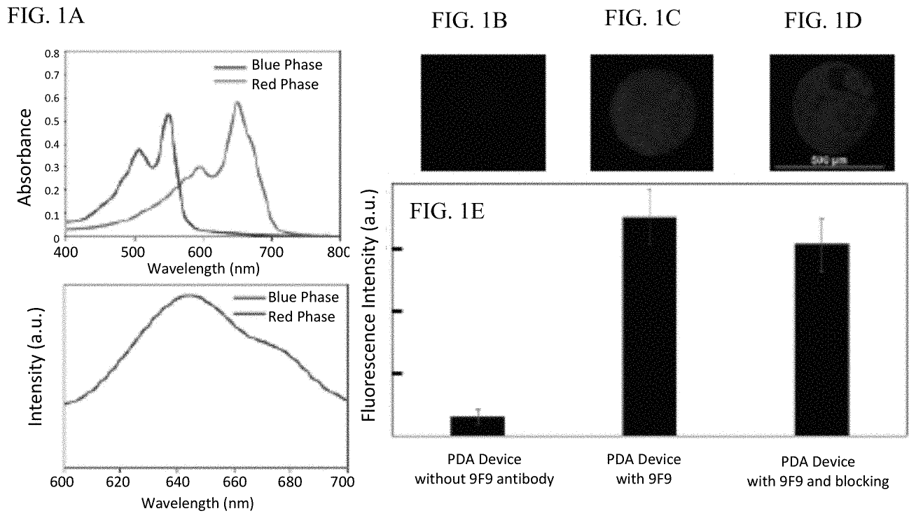

PDA liposome was immobilized on the glass substrate to develop the solid type sensor as described above. To confirm successful immobilization of PDA-epoxy liposomes onto the amine-modified glass substrate, a PDA-epoxy liposome solution was incubated on the amine substrate, photopolymerized, and heat was applied to the immobilized PDA liposomes at 150° C. for 3 mins. This effectively distorted the resulting conjugated PDA backbone and produced red fluorescence. One could observe the saturated fluorescence intensity of red-phase PDA by using a fluorescence microscope (FIG. 1A). There was only 3.45% of the average difference in fluorescence intensity across 36 devices, indicating the robust and homogenous immobilization of the PDA liposome onto the glass substrate as well as the reproducibility of the devices.

9F9 antibodies were subsequently tethered to the immobilized PDA liposome layer on the amine substrates followed by blocking any unreacted epoxy groups of the PDA layer with ethanolamine. Further testing was implemented to confirm this blocking does not affect the efficacy of the antibody before applying whole blood samples on the developed PDA liposome-9F9 microarray. As shown in the FIG. 1B, upon incubating with the whole blood samples, no signal was detected from the PDA microarray device without having 9F9 antibodies but simply blocked with ethanolamine. On the contrary, as shown in the FIG. 1D, antibody-incubated and ethanolamine-blocked device emitted red fluorescence signal from the samples. The device having 9F9 antibodies without the enthanolmaine blocking (FIG. 1C) showed 13.72% higher fluorescence signal intensity than the ethanolamine-blocked device (FIG. 1E). It is contemplated that that non-specific binding of non-targeted blood components to unreacted epoxy groups resulted in additional random background signal.

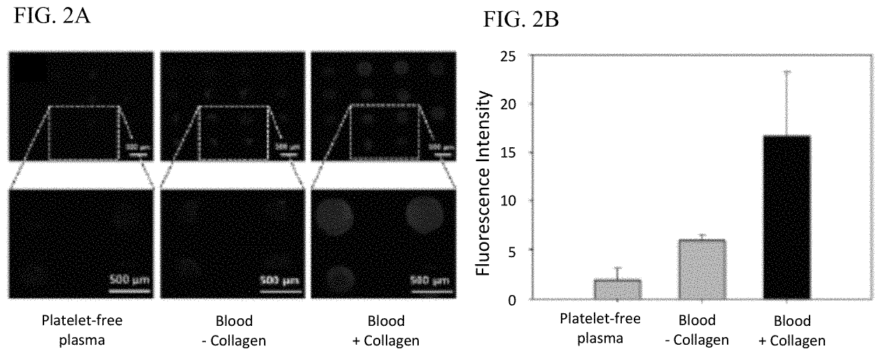

The two major receptors on the platelet surface, integrin α₂β₁ and GPVI, are used for interaction with collagen, which activates platelet. Additionally, adenosine diphosphate (ADP) and epinephrine activate platelets which are mediated by 3 purinergic receptors (P2Y1, P2Y12, and P2X1) and the alpha2 adrenergic receptor, respectively. Platelet activation then leads to the increased affinity of the GPIIb/IIIa to plasma fibrinogen molecule leading to platelet aggregation.^{32, 33} The PDA microarray device was developed to selectively measure activated platelets since the 9F9 antibody tethered on the PDA surface selectively interacts with fibrinogen bound to the activated platelets. In order to ensure specific measurement for activated platelets, we conducted a control experiment comparing whole blood samples with and without 40 mg/ml of collagen. As seen in FIG. 2, the PDA device with added collagen displayed a red fluorescence intensity of 2.8 times greater than the device without it. The significant increase in the fluorescence signal of the sample with collagen confirmed that our PDA liposome-9F9 microarray device detected specifically for activated platelets and was unaffected by the number of total platelets present in the samples.

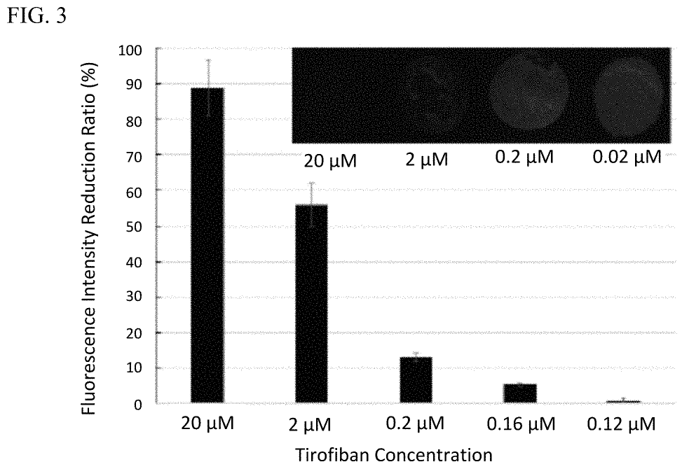

The devised PDA microarray activated platelets. Due to its specificity, it was further investigated whether the device can be used to determine appropriate dosage of antiplatelet drugs for individual patients and their personal healthcare providers. Tirofiban is an inhibitor of platelet-activated coagulation which acts by inhibiting GPIIb/IIIa. It was incubated in the blood samples for 20 mins. The results showed that the Tirofiban concentration was inversely related to the fluorescence signal intensity of the PDA microarray device (FIG. 3). As the concentration of incubated Tirofiban increases in the sample, the signal decreases. The limit of detection of Tirofiban in the sample was measured to be 0.16 μM.

The 20-mins incubation time of the anticoagulation drug was selected to obtain the optimal signal. 100 ml of the blood samples were incubated with various Tirofiban concentrations to 9 mm diameter silicon isolator of the PDA microarray device. The red fluorescence signal observed for all samples significantly increased when Tirofiban was incubated more than 30 minutes. After 50 min, even the blood sample having 20 ml of Tirofiban showed a similar signal intensity to samples without Tirofiban. Altogether, it was observed that in the open-air condition with longer than 30-mins incubation time, signal intensities from both Tirofiban-treated group and non-treated group showed similar level of coagulation from activated platelets. Therefore, it was concluded that the anticoagulation drug should be incubated for 20 mins for the best reliable and reproducible result to be presented in the PDA microarray device.

To confirm the results from the PDA liposome-9F9 microarray, a modified flow cytometric assay was conducted using the same anti-human fibrinogen antibody, 9F9. FIG. 4 shows the flow cytometric analysis (A-F) of the inhibition of collagen and epinephrine stimulated human platelets by Tirofiban, collagen concentration responses on 9F9 antibody expression (G), and the Tirofiban's inhibition of 40 mg/ml collagen and 10 mM epinephrine-stimulated human platelets (H). This stimulation of platelets elicited a maximum platelet activation response.

The results of the modified flow cytometric assay confirmed the results of the PDA liposome-9F9 microarray. The level of Tirofiban inhibition directly affects the measured fluorescence intensity of FACS analysis since the fluorescence intensity is determined by the level of bound F9F antibody-FITC conjugate to platelets. Less bound F9F conjugate to platelets means an increase in the inhibition of platelet activation. Flow cytometry analysis data shown in FIG. 4G demonstrates that 9F9 antibody expression increases as the concentration of collagen increases, supporting the results shown in FIG. 2. As shown in FIG. 4H, the increase in Tirofiban concentration resulted in an increase in the % inhibition of platelet activation via collagen stimulation. The Tirofiban inhibited collagen with IC₅₀ values of about 0.0015 nM. Therefore, direct detection of platelet activation using the specific antibody, 9F9, to platelet-bound fibrinogen allows the determination of the extent of platelet activation under most conditions including contact activation in extracorporeal circulations, antiplatelet drug effects and hereditary platelet diseases. Similarly, as shown in FIG. 3, the PDA liposome-9F9 complex produced weaker fluorescence when the concentration of Tirofiban was increased. Thus, the PDA liposome-9F9 assay provides a simple single-step measurement procedure compared to the complex flow cytometric method to assess the real-time functional state of circulating platelets.

### REFERENCES

1. Femia, E. A.; Scavone, M.; Lecchi, A.; Cattaneo, M., Effect of platelet count on platelet aggregation measured with impedance aggregometry (Multiplate (TM) analyzer) and with light transmission aggregometry. J Thromb Haemost 2013, 11 (12), 2193-2196.
2. Michelson, A. D.; Barnard, M. R.; Krueger, L. A.; Frelinger, A. L.; Furman, M. I., Evaluation of platelet function by flow cytometry. Methods 2000, 21 (3), 259-270.
3. Karagkiozaki, V.; Logothetidis, S.; Kalfagiannis, N.; Lousinian, S.; Giannoglou, G., Atomic force microscopy probing platelet activation behavior on titanium nitride nanocoatings for biomedical applications. Nanomed-Nanotechnol 2009, 5 (1), 64-72.
4. Chen, X. Q.; Zhou, G. D.; Peng, X. J.; Yoon, J., Biosensors and chemosensors based on the optical responses of polydiacetylenes. Chem Soc Rev 2012, 41 (13), 4610-4630.
5. Ahn, D. J.; Kim, J. M., Fluorogenic polydiacetylene supramolecules: Immobilization, micropatterning, and application to label-free chemosensors. Accounts Chem Res 2008, 41 (7), 805-816.
6. Seo, S.; Shanker, A.; Kwon, M.; Kim, J., Functional Polydiacetylene Liposomes as a Self-Signaling and Signal-Amplifying Bio- and Chemical Sensor and Sensor Array. Liposomes in Analytical Methodologies 2016, 167-201.
7. Lu, Y.; Yang, Y.; Sellinger, A.; Lu, M.; Huang, J.; Fan, H.; Haddad, R.; Lopez, G.; Burns, A. R.; Sasaki, D. Y.; Shelnutt, J.; Brinker, C. J., Self-assembly of mesoscopically ordered chromatic polydiacetylene/silica nanocomposites. Nature 2001, 410,
8. Okada, S.; Peng, S.; Spevak, W.; Charych, D., Color and chromism of polydiacetylene vesicles. Accounts Chem Res 1998, 31 (5), 229-239.
9. Park, I. S.; Park, H. J.; Kim, J. M., A Soluble, Low-Temperature Thermochromic and Chemically Reactive Polydiacetylene. Acs Appl Mater Inter 2013, 5 (17), 8805-8812.
10. Seo, S.; Lee, J.; Kwon, M. S.; Seo, D.; Kim, J., Stimuli-Responsive Matrix-Assisted Colorimetric Water Indicator of Polydiacetylene Nanofibers. Acs Appl Mater Inter 2015, 7 (36), 20342-20348.
11. Lee, J.; Pyo, M.; Lee, S. H.; Kim, J.; Ra, M.; Kim, W. Y.; Park, B. J.; Lee, C. W.; Kim, J. M., Hydrochromic conjugated polymers for human sweat pore mapping. Nat Commun 2014, 5, 3736.
12. Kang, D. H.; Jung, H. S.; Ahn, N.; Yang, S. M.; Seo, S.; Suh, K. Y.; Chang, P. S.; Jeon, N. L.; Kim, J.; Kim, K., Janus-Compartmental Alginate Microbeads Having Polydiacetylene Liposomes and Magnetic Nanoparticles for Visual Lead(II) Detection. Acs Appl Mater Inter 2014, 6 (13), 10631-10637.
13. Lee, J.; Kim, H. J.; Kim, J., Polydiacetylene liposome arrays for selective potassium detection. J Am Chem Soc 2008, 130 (15), 5010-5011.
14. Lee, J.; Jun, H.; Kim, J., Polydiacetylene-Liposome Microarrays for Selective and Sensitive Mercury(II) Detection. Adv Mater 2009, 21 (36), 3674-3677.
15. Lee, J.; Chang, H. T.; An, H.; Ahn, S.; Shim, J.; Kim, J. M., A protective layer approach to solvatochromic sensors. Nat Commun 2013, 4, 2461.
16. Lee, J.; Seo, S.; Kim, J., Colorimetric Detection of Warfare Gases by Polydiacetylenes Toward Equipment-Free Detection. Advanced Functional Materials 2012, 22 (8), 1632-1638.
17. Kang, D. H.; Jung, H. S.; Ahn, N.; Lee, J.; Seo, S.; Suh, K. Y.; Kim, J.; Kim, K., Biomimetic detection of aminoglycosidic antibiotics using polydiacetylene-phospholipids supramolecules. Chem Commun 2012, 48 (43), 5313-5315.
18. Lee, J.; Jeong, E. J.; Kim, J., Selective and sensitive detection of melamine by intra/inter liposomal interaction of polydiacetylene liposomes. Chem Commun 2011, 47 (1), 358-360.
19. Kolusheva, S.; Boyer, L.; Jelinek, R., A colorimetric assay for rapid screening of antimicrobial peptides. Nat Biotechnol 2000, 18 (2), 225-227.
20. Kauffman, J. S.; Ellerbrock, B. M.; Stevens, K. A.; Brown, P. J.; Pennington, W. T.; Hanks, T. W., Preparation, Characterization, and Sensing Behavior of Polydiacetylene Liposomes Embedded in Alginate Fibers. Acs Appl Mater Inter 2009, 1 (6), 1287-1291.
21. Xu, Q.; Lee, S.; Cho, Y.; Kim, M. H.; Bouffard, J.; Yoon, J., Polydiacetylene-Based Colorimetric and Fluorescent Chemosensor for the Detection of Carbon Dioxide. J Am Chem Soc 2013, 135 (47), 17751-17754.
22. Kang, D. H.; Jung, H. S.; Lee, J.; Seo, S.; Kim, J.; Kim, K.; Suh, K. Y., Design of Polydiacetylene-Phospholipid Supramolecules for Enhanced Stability and Sensitivity. Langmuir 2012, 28 (19), 7551-7556.
23. Gou, M. L.; Qu, X.; Zhu, W.; Xiang, M. L.; Yang, J.; Zhang, K.; Wei, Y. Q.; Chen, S. C., Bio-inspired detoxification using 3D-printed hydrogel nanocomposites. Nature Communications 2014, 5, 3774.
24. Seo, S.; Lee, J.; Choi, E. J.; Kim, E. J.; Song, J. Y.; Kim, J., Polydiacetylene Liposome Microarray Toward Influenza A Virus Detection: Effect of Target Size on Turn-On Signaling. Macromol Rapid Comm 2013, 34 (9), 743-748.
25. Jung, S. H.; Jang, H.; Lim, M. C.; Kim, J. H.; Shin, K. S.; Kim, S. M.; Kim, H. Y.; Kim, Y. R.; Jeon, T. J., Chromatic Biosensor for Detection of Phosphinothricin Acetyltransferase by Use of Polydiacetylene Vesicles Encapsulated within Automatically Generated Immunohydrogel Beads. Anal Chem 2015, 87 (4), 2072-2078.
26. Gill, I.; Ballesteros, A., Immunoglobulin-polydiacetylene sol-gel nanocomposites as solid-state chromatic biosensors. Angew Chem Int Edit 2003, 42 (28), 3264-3267.
27. Park, C. H.; Kim, J. P.; Lee, S. W.; Jeon, N. L.; Yoo, P. J.; Sim, S. J., A Direct, Multiplex Biosensor Platform for Pathogen Detection Based on Cross-linked Polydiacetylene (PDA) Supramolecules. Advanced Functional Materials 2009, 19 (23), 3703-3710.
28. Charych, D. H.; Nagy, J. O.; Spevak, W.; Bednarski, M. D., Direct Colorimetric Detection of a Receptor-Ligand Interaction by a Polymerized Bilayer Assembly. Science 1993, 261 (5121), 585-588.
29. Kang, D. H.; Jung, H. S.; Kim, K.; Kim, J., Mussel-Inspired Universal Bioconjugation of Polydiacetylene Liposome for Droplet-Array Biosensors. Acs Appl Mater Inter 2017, 9 (48), 42210-42216.
30. Frojmovic, M. M.; Mooney, R. F.; Wong, T., Dynamics of Platelet Glycoprotein Iib-Iiia Receptor Expression and Fibrinogen Binding 0.1. Quantal Activation of Platelet Subpopulations Varies with Adenosine-Diphosphate Concentration. Biophys J1994, 67 (5), 2060-2068.
31. Frojmovic, M. M.; Mooney, R. F.; Wong, T., Dynamics of Platelet Glycoprotein Iib-Iiia Receptor Expression and Fibrinogen Binding 0.2. Quantal Activation Parallels Platelet Capture in Stir-Associated Microaggregation. Biophys J1994, 67 (5), 2069-2075.
32. Farndale, R. W., Collagen-induced platelet activation. Blood Cell Mol Dis 2006, 36 (2), 162-165.
33. Inoue, 0.; Suzuki-Inoue, K.; Dean, W. L.; Frampton, J.; Watson, S. P., Integrin alpha(2)beta(1) mediates outside-in regulation of platelet spreading on collagen through activation of Src kinases and PLC-gamma 2. J Cell Biol 2003, 160 (5), 769-780.
34. Abrams, C. S.; Ellison, N.; Budzynski, A. Z.; Shattil, S. J., Direct detection of activated platelets and platelet-derived microparticles in humans. Blood 1990, 75 (1), 128-138.

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in molecular biology, in vitro fertilization, development, or related fields are intended to be within the scope of the following claims.

We claim:

1. A method, comprising:
a) contacting a sample comprising platelets with a device comprising a solid surface comprising an array of polydiacetylene (PDA)-liposomes conjugated to an antibody that specifically binds to activated platelets; and
b) detecting activated platelets bound to said antibody.

2. The method of claim 1, wherein said PDA is 10,12-pentacosadiynoic acid (PCDA)-epoxy.

3. The method of claim 1, wherein said liposome comprises 1,2-dimyristoyl-sn-glycero-3-phosphate (DMPA).

4. The method of claim 1, wherein said solid support is glass.

5. The method of claim 1, wherein said solid support is modified.

6. The method of claim 5, wherein said modification is amine modification.

6. The method of claim 1, wherein said antibody is 9F9.

7. The method of claim 1, wherein said detecting comprises detecting fluoresce emitting from said device.

8. The method of claim 1, wherein said antibody binds to fibrinogen on activated platelets.

9. The method of claim 1, wherein said detecting is quantitative.

10. The method of claim 9, wherein said quantitative detecting comprises comparing the level of fluorescence to a standard level.

11. The method of claim 10, wherein said standard level is indicative of a threshold level of activation of said platelets.

12. The method of claim 1, wherein said blood sample is from a subject.

13. The method of claim 12, wherein said subject has been administered an anti-platelet drug.

14. The method of claim 13, wherein said detecting is repeated one or more times during said administration.

15. The method of claim 13, wherein said detecting is used to determine a treatment course of action.

16. The method of claim 15, wherein said treatment course of action comprises one or more actions selected from the group consisting of changing the dose of said anti-platelet drug, changing said anti-platelet drug, and stopping said anti-platelet drug.

17. The method of claim 12, wherein said anti-platelet drug is a test compound.

18. The method of claim 1, wherein said blood sample is contacted with an anti-platelet drug prior to said detecting.

19. The method of claim 1, wherein said blood sample is whole blood.

20. A device comprising a solid surface comprising an array of polydiacetylene (PDA)-liposomes conjugated to an antibody that specifically binds to activated platelets.
